(19) United States
(12) Patent Application Publication
(10) Pub. No.: US 2023/0162019 A1
(43) Pub. Date: May 25, 2023

(54) TOPOLOGICAL SIGNATURES FOR DISEASE CHARACTERIZATION

(71) Applicant: International Business Machines Corporation, Armonk, NY (US)

(72) Inventors: Laxmi Parida, Mohegan Lake, NY (US); Aldo Guzman Saenz, White Plains, NY (US); Sayan Mandal, Santa Fe, NM (US); Niina Haiminen, Valhalla, NY (US)

(21) Appl. No.: 17/533,266

(22) Filed: Nov. 23, 2021

(51) Int. Cl. G06N 3/08; G16H 70/60; G16H 50/20

(52) U.S. Cl. CPC G06N 3/08; G16H 70/60; G16H 50/20

(57) ABSTRACT

Gene expression data associated with a subject can be received. Pair-wise similarities between genes in the gene expression data can be determined. The gene expression data can be transformed into topological summaries based on the pair-wise similarities. A neural network can be trained using a training set created based on the topological summaries. A new sample can be received and input to the neural network, where the neural network can predict the new sample's phenotype.

### BACKGROUND

The present application relates generally to computers and computer applications, and more particularly to machine learning, neural networks, creating training sets for training machine learning model, and disease detection.

Machine learning allows computers or machines to perform tasks without being explicitly programmed to perform the tasks. The computers or machines, for example, are enabled to learn from experience in performing a task, for example making classifications or predictions.

### BRIEF SUMMARY

The summary of the disclosure is given to aid understanding of a computer system and method of generating topological signatures for disease characterization and/or training of machine learning models based on such topological signatures, and not with an intent to limit the disclosure or the invention. It should be understood that various aspects and features of the disclosure may advantageously be used separately in some instances, or in combination with other aspects and features of the disclosure in other instances. Accordingly, variations and modifications may be made to the computer system and/or their method of operation to achieve different effects.

A computer-implemented method of training a neural network for disease detection in a sample can be provided. The method, in an aspect, can include receiving gene expression data associated with a subject. The method can also include determining pair-wise correlations similarities between genes in the gene expression data. The method can also include transforming the gene expression data into topological summaries based on the pair-wise similarities. The method can also include training a neural network using a training set created based on the topological summaries.

A system, in an aspect, can include a processor and a memory device coupled with the processor. The processor can be configured to receive gene expression data associated with a subject. The processor can also be configured to determine pair-wise similarities between genes in the gene expression data. The processor can also be configured to transform the gene expression data into topological summaries based on the pair-wise similarities. The processor can also be configured to train a neural network using a training set created based on the topological summaries.

A computer readable storage medium storing a program of instructions executable by a machine to perform one or more methods described herein also may be provided.

Further features as well as the structure and operation of various embodiments are described in detail below with reference to the accompanying drawings. In the drawings, like reference numbers indicate identical or functionally similar elements.

### BRIEF DESCRIPTION OF THE DRAWINGS

FIG. 1 is a diagram illustrating an overview of a pipeline in an embodiment.

FIG. 2 shows input data in an embodiment in the form of a table of rows and columns.

FIG. 3 shows example of new data, which can be transformed into topological summaries for prediction in an embodiment.

FIG. 4 is a flow diagram illustrating a method in an embodiment.

FIG. 5 is a diagram showing components of a system in one embodiment that can train and/or run a neural network or another machine learning model for disease detection, prediction or classification.

FIG. 6 illustrates a schematic of an example computer or processing system that may implement a system according to one embodiment.

FIG. 7 illustrates a cloud computing environment in one embodiment.

FIG. 8 illustrates a set of functional abstraction layers provided by cloud computing environment in one embodiment of the present disclosure.

### DETAILED DESCRIPTION

In one or more embodiments, systems, methods and techniques can be provided for building or supporting a pipeline to process biological data like gene expressions, to output topological summaries of the biological data, which can be used to train one or more machine learning models to predict or classify potential disease in a given sample. The pipeline can also include training of one or more learning models, for example, based on topological summaries. For instance, phenotype prediction can be performed using topological data analysis pipeline. For example, in one or more embodiments, the generated topological summaries of diseased and healthy biological data can be input to one or more machine learning models such as neural networks to train such machine learning models to predict or classify whether a given sample presents certain phenotype, e.g., is diseased or healthy. Examples of topological summaries can include but are not limited to, bar diagrams, heat maps, and/or other signatures characterizing the biological data.

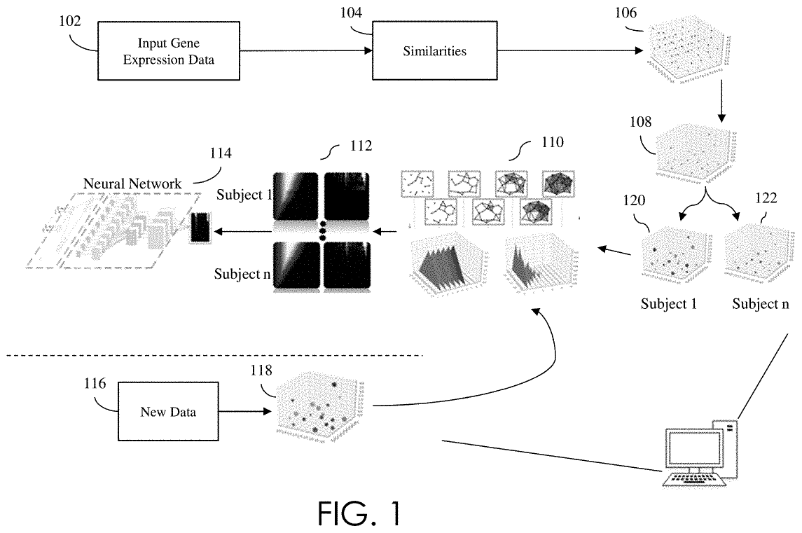

FIG. 1 is a diagram illustrating an overview of a pipeline in an embodiment. The components or functionalities shown in the figure can be implemented on one or more computer processors including, e.g., one or more hardware processors. One or more hardware processors, for example, may include components such as programmable logic devices, microcontrollers, memory devices, and/or other hardware components, which may be configured to perform respective tasks described in the present disclosure. Coupled memory devices may be configured to selectively store instructions executable by one or more hardware processors. A processor or hardware processor may be a central processing unit (CPU), a graphics processing unit (GPU), a field programmable gate array (FPGA), an application specific integrated circuit (ASIC), another suitable processing component or device, or one or more combinations thereof. The processor may be coupled with a memory device. The memory device may include random access memory (RAM), read-only memory (ROM) or another memory device, and may store data and/or processor instructions for implementing various functionalities associated with the methods and/or systems described herein. The processor may execute computer instructions stored in the memory or received from another computer device or medium.

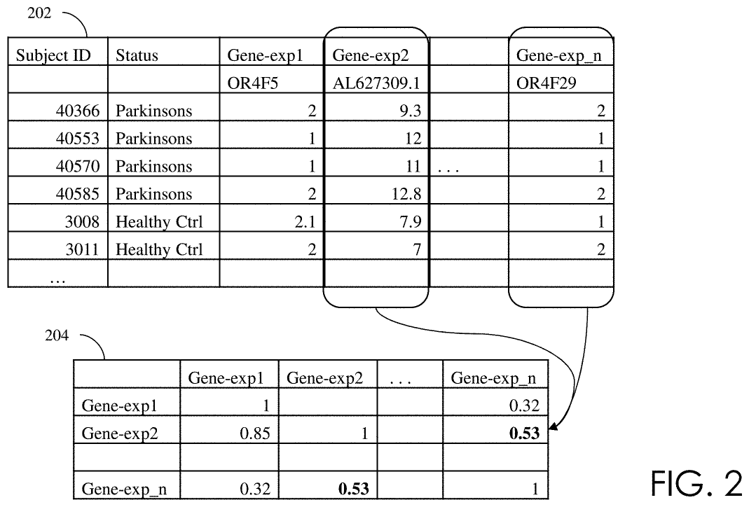

In an embodiment, a system and/or method disclosed herein can characterize subjects with topological signatures based on their biomarker measurements. Input 102 can be received or obtained, which includes biological or biomarker data such as gene expression of subjects with disease and without disease. An example of such input data is shown in FIG. 2. In FIG. 2, input data 202 shown is in the form of a matrix or table of rows and columns. In the example matrix 202, the rows include subjects (e.g., patients) and the columns includes genes. The data can also be normalized, for example, with Robust Differential Gene Expression (RoDEO). Based on such input data, the system and/or method can output topological signature (e.g., bar diagram, heatmap) per subject. In an embodiment, the received input data can be normalized data. For example, the input data 102 can be normalized gene expression counts for patients and healthy controls. In another embodiment, the system and/or method may receive the input data (which may not be normalized) and normalize the input data.

Referring to FIG. 1, at 104, correlation or similarity between all pairs of features can be computed. In an embodiment, this similarity computation can be performed with all subjects in training set at the same time, e.g., for each pair of genes there is a single distance correlation or similarity for the entire training set. For instance, pair-wise distances between all features (e.g., genes) can be computed. In an embodiment, the similarity computation can use a metric, e.g., a function satisfying certain properties that takes two points and returns a real number. In an embodiment, the similarity computation can use Euclidian distance measure. For instance, a processor may compute similarity across columns of matrix shown in FIG. 2. In an embodiment, the processor may embed the metrics or measurements specified in the columns into Euclidean space, for instance, for resampling. In an embodiment, if resampling is not needed or not to be performed, this embedding into Euclidean space can be skipped, and the processor may work in metric space. In an aspect, the Euclidian space is a vector space. In an aspect, the metric space need not always be a vector space. FIG. 2 at 204 shows an example of pair-wise distance correlation between all genes in an embodiment, for example, in the training set associated with a plurality of subjects.

Referring to FIG. 1, as shown at 106, weighted point cloud can be produced for each sample by assigning weights of nearest gene in the sample for that subject. A point cloud refers to a set of data points in space. For example, the points may represent a shape, e.g., a 3-dimensional (3D) shape, where each point position has its set of Cartesian coordinates. For example, for building a point cloud, points can be assigned to vertices based on the values (e.g., data in the columns shown in FIG. 2). A weighted point cloud in this context is a set of points with a function that maps each point to a real number. In an embodiment, for each subject, a weighted point cloud can be obtained by assigning to each point (which is a feature) the value of that feature for that subject.

Optionally, resampling or sub-sampling can be performed as shown at 108, which may produce better estimate of manifold. For example, in an embodiment, if N is the total number of features and S is the target number of subsampled/resampled features, subsampling may be performed by randomly selecting a subset of {1, . . . , N} of size S, and selecting features at those indices. In an embodiment, resampling, e.g., if features are in Euclidean space, may be performed by enveloping each point by a ball of radius r and sampling S points from the union of those balls. For example, data can be embedded in R\hat{ }n as an optional step for making the processing at 108 feasible on general metric data (R\hat{ }n here denotes the canonical n-dimensional vector space over real numbers). Multi-dimensional scaling can be performed. Resampling used to examine gene expression can further aid in computation of topological summaries in practice. Resampling may obtain, with a guaranteed probability, a sampled point cloud resembling the original shape. An example resampled or subsampled data in point cloud is shown at 108. The multidimensional point cloud at 108 shows an example of balanced re-sampling in an embodiment. Multidimensional point clouds at 120 and 122 show weighted point clouds based on individual subject gene expression. For instance, a weighted point cloud based on individual subject gene expression is generated per the individual subject (e.g., subject 1 . . . subject n, where there are n number of subjects). For example, 108 shows example resampled or subsampled point cloud, and 120 and 122 represent those point clouds with weights associated to their elements.

At 110, topological data analysis (TDA) is performed on the weighted point cloud to generate, per subject, persistence landscapes, e.g., one for each homology degree. For instance, genomic or gene expression data for multiple phenotypes can be converted to TDA features. This mechanism can characterize a subject in terms of the subject's biomarker data in the context of a larger population. Example topological summaries are shown at 112. A persistence landscape is an instance or example of a topological summary. In an embodiment, there can be one persistence landscape, that can have a component for each homology degree. In an embodiment, a single object can have several persistence landscapes, one per homology degree, in contrast with a single landscape with several homology degree components, but both expressions can have the same meaning. In an embodiment, for a given topological space X, its homology is an algebraic construction that captures some of its topological information. In an embodiment, a methodology disclosed herein can consider persistent simplicial homology with coefficients in a finite field (e.g., the field with two elements), which can be viewed as instances of vector spaces and linear transformations among them. For instance, in an embodiment, such methodology can include constructing a weighted Vietoris-Rips complex C from the weighted point cloud; computing C's persistent homology, thus obtaining a family of vector spaces and linear transformations; computing the barcode (e.g., a description of certain generators in the family of the previous step); and from the barcode or based on the barcode, computing the persistence landscape. In an embodiment, this can be performed for all relevant degrees. Homology degree herein refers to the dimensionality of the features that are being characterized: e.g., degree 0 means connected components, degree 1 means looking at edges and triangles to detect “holes”, degree 2 means looking at triangles and tetrahedra to detect “holes” of dimension 2, and similarly for higher degrees.

The system and/or method (e.g., a computer processor implementing or running the system and/or method described herein) may also quantize or vectorize the persistence landscapes, e.g., the TDA features or topological summaries. For instance, a processor may convert the topological summaries into tensors that can be fed into the neural network. In an embodiment, a persistence landscape for each degree can be a collection of functions fi(x)=y from real numbers to real numbers. In an embodiment, a methodology disclosed herein can quantize the values in both domain and co-domain so that there are a finite number of possible values for x and y. The quantized values and considering that there can be finitely many functions fi allow to be able to consider triples (i, x, y) of floating numbers. These fit in an I times X times Y—shaped tensor, where I is the total number of functions, X the total number of possible x values and Y is the total number of possible y values. When all degrees are considered at the same time, 4-tuples (i, x, y, d) can be considered and thus there can be a tensor of shape I times X times Y times D, where D is the total number of degrees considered.

Shown at 114, vectorization of persistence landscapes (e.g., tensor) is fed to a prediction method such as a convolutional neural network (CNN) to train a CNN model for separating healthy and affected (e.g., diseased) samples. For instance, the TDA features can be converted to tensors based on homology to feed into a CNN. The model, which can be built on a population (e.g., training data that includes biomarkers of population of subjects) can be used to predict the phenotype, or characterizing, of a new sample.

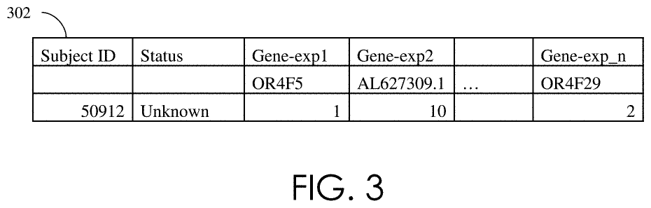

At 116, given a new sample, a weighted point cloud 118 can be generated of the new sample, for example, using the processing shown at 104, 106 and optionally 108, and topological data analysis as shown at 110 can be performed on the generated weighted point cloud of the new sample to generate a persistence landscape corresponding to the new sample. This persistence landscape can be input to the trained model (e.g., the CNN model trained at 112), for CNN model to predict the phenotype or characterization (e.g., healthy or affected) associated with the new sample. The system and/or method can add to understanding of disease mechanism and advance individualized medicine, improving phenotype prediction and/or explaining characteristics of a phenotype. FIG. 3 shows example of new data 302, which can be converted to point cloud 118 (FIG. 1) in an embodiment. For instance, gene expression weights can be applied on the new data 302 to generate the point cloud 118. More specifically, consider gene expression matrix denoted by M, where there are samples {S_1, . . . , S_m} with genes {G_1, . . . , G_n}. M_ij can be referred to as the gene expression value of gene G_j at sample S_i. For a given sample S_i, the system and/or method in an embodiment can set the weights of the point cloud to be the values at the i-th row of M. Since these weights depend on each S_i, there can be for each sample S_i a weighted point cloud. Similarly, if the sample is not part of the original data matrix but its gene expression values are provided, the system and/or method can set the weights of the point cloud to be the sample's gene expression values.

In an embodiment, the system and/or method creates a pipeline for processing biomarker data and outputting a topological summary. Such topological summary can characterize a subject in terms of the subject's biomarker data in the context of a larger population. A model such as a neural network built on a population can be applied to predict the phenotype of a new sample. In an aspect, re-sampling can be used to examine gene expression to further aid in computation of topological summaries. In an aspect, a sampled point cloud resembling the original shape can be obtained.

In an aspect, topological summaries allow for using features that are relevant for a particular prediction, allowing a machine (machine learning model) to decide whether a sample is healthy or not and/or why the machine made that prediction. Since patient data is large dimensional, the system and/or method disclosed herein can help in discerning what is relevant to the disease and what is not relevant to the disease. In an aspect, the system and/or method can generate a signature in terms of topology (e.g., also referred to as a topological signature or topological summary) that can be used to identify or characterize a patient, e.g., whether diseased or healthy based on biomarkers or pathways of that patient. In an aspect, the system and/or method may include feeding a machine learning model a number of healthy samples and a number of diseased samples, allowing the machine learning model to learn from the samples. Given a new sample, the trained machine learning model can predict or characterize the new sample as healthy or diseased, e.g., a particular disease. For instance, topological signatures are generated and input to the machine learning model to train based on the topological signatures. Training data and new unseen data provided to the machine learning model can be at topological level.

In an aspect, the system and/or method may subsample data sets to approximate it. In another aspect, the system and/or method may resample data sets assuming some distribution of data. An example of distribution of data can be to consider a mixture of Gaussians of a given mean and standard deviation centered around each point in the original dataset. For instance, instead of taking a subset of original points, the system and/or method may replace those points by better ones while keeping the topology, e.g., the topology is preserved under this resampling.

The system and/or method described herein can be applied in diagnostics, e.g., diagnosing Parkinson's disease versus healthy prediction, and/or other disease or medical conditions. The system and/or method described herein can also be used in selecting a treatment option, for example, based on a diagnoses. The system and/or method described herein can further be used in estimating disease progression and predicting outcomes.

In an embodiment, the system and/or method described herein use persistent homology in a pipeline of processing biomarker data in outputting topological summaries. The pipeline can achieve phenotype prediction from gene expression data. In an embodiment, a process on gene expression data uses distance similarities or correlations and subsampling to compute tensors from topological summaries, and use the tensors for phenotype prediction.

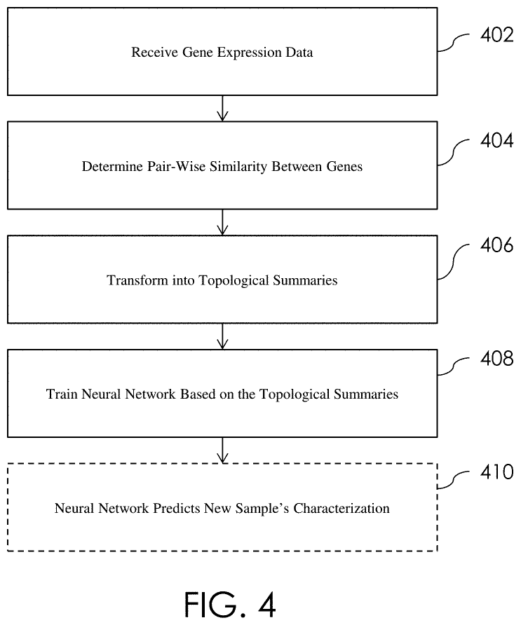

FIG. 4 is a flow diagram illustrating a method in an embodiment. The method can be implemented by one or more hardware processors, and/or run on one or more hardware processors. At 402, gene expression data associated with a subject is received. Multiples of sets of gene expression data can be received associated with multiple subject, e.g., a set of gene expression data per subject.

At 404, pair-wise similarities between all genes in the gene expression data are determined. For example, for all combinations of pairs of genes in the gene expression data, pair-wise similarities can be built or determined. In an embodiment, a distance measure between a pair of genes can be computed as a pair-wise correlation.

At 406, the set of gene expression data is transformed into topological summaries, for example, based on the pair-wise similarities. In an embodiment, the pair-wise similarities are used to create a point cloud, the point cloud used to transform the gene expression data into the topological summaries. In an embodiment, resampling data points can be performed based on the gene expression data, e.g., the pair-wise similarities, where the resampled data points are transformed into the topological summaries. In an embodiment, subsampling data points can be performed based on the gene expression data, e.g., the pair-wise similarities, where the subsampled data points are transformed into the topological summaries.

At 408, based on the topological summary, e.g., a training set created based on the topological summaries, a neural network is trained to predict a characterization of a given sample. In an embodiment, the neural network can include a convolutional neural network. Other types of machine learning, deep learning and/or neural networks can be used.

At 410, the trained neural network is fed a new sample, e.g., new or unseen gene expression data, where the neural network predicts the new sample's characterization. For instance, the new sample can include gene expression data previously unseen. Such data can be transformed into topological summaries, based on which the neural network can perform its prediction or classification. In an embodiment, the topological summaries can be converted into a tensor, and the tensor can be fed into the neural network.

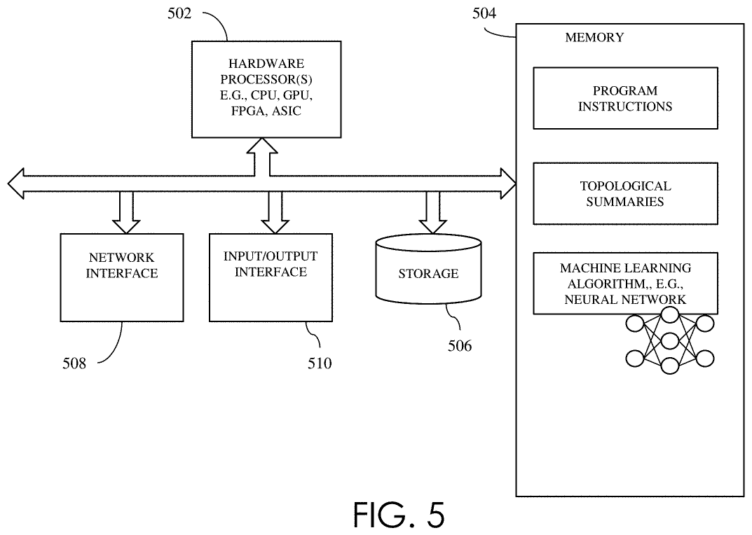

FIG. 5 is a diagram showing components of a system in one embodiment that can train a neural network or another machine learning model for disease detection, prediction or classification. One or more hardware processors 502 such as a central processing unit (CPU), a graphic process unit (GPU), and/or a Field Programmable Gate Array (FPGA), an application specific integrated circuit (ASIC), and/or another processor, may be coupled with a memory device 504, and may train a neural network or another machine learning model to characterize a sample gene expression data. A memory device 504 may include random access memory (RAM), read-only memory (ROM) or another memory device, and may store data and/or processor instructions for implementing various functionalities associated with the methods and/or systems described herein. One or more processors 502 may execute computer instructions stored in memory 504 or received from another computer device or medium. A memory device 504 may, for example, store instructions and/or data for functioning of one or more hardware processors 502, and may include an operating system and other program of instructions and/or data. One or more hardware processors 502 may receive gene expression data associated with a subject. One or more hardware processors 502 may determine pair-wise similarities between genes in the gene expression data. One or more hardware processors 502 may transform the gene expression data into topological summaries based on the pair-wise similarities. One or more hardware processors 502 may train a neural network using a training set created based on the topological summaries. Input gene expression data may be stored in a storage device 506 or received via a network interface 508 from a remote device, and may be temporarily loaded into a memory device 504 for building or generating a prediction model. The learned prediction model may be stored on a memory device 504, for example, for running by one or more hardware processors 502. One or more hardware processors 502 may be coupled with interface devices such as a network interface 508 for communicating with remote systems, for example, via a network, and an input/output interface 510 for communicating with input and/or output devices such as a keyboard, mouse, display, and/or others.

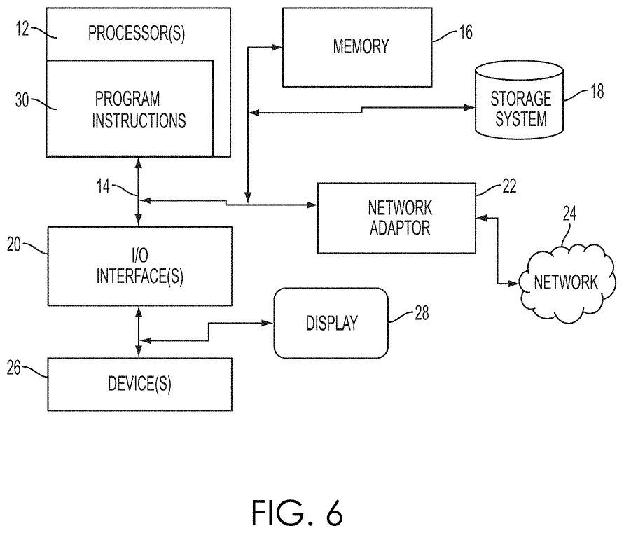

FIG. 6 illustrates a schematic of an example computer or processing system that may implement a system in one embodiment. The computer system is only one example of a suitable processing system and is not intended to suggest any limitation as to the scope of use or functionality of embodiments of the methodology described herein. The processing system shown may be operational with numerous other general purpose or special purpose computing system environments or configurations. Examples of well-known computing systems, environments, and/or configurations that may be suitable for use with the processing system shown in FIG. 6 may include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

The computer system may be described in the general context of computer system executable instructions, such as program modules, being run by a computer system. Generally, program modules may include routines, programs, objects, components, logic, data structures, and so on that perform particular tasks or implement particular abstract data types. The computer system may be practiced in distributed cloud computing environments where tasks are performed by remote processing devices that are linked through a communications network. In a distributed cloud computing environment, program modules may be located in both local and remote computer system storage media including memory storage devices.

The components of computer system may include, but are not limited to, one or more processors or processing units 12, a system memory 16, and a bus 14 that couples various system components including system memory 16 to processor 12. The processor 12 may include a module 30 that performs the methods described herein. The module 30 may be programmed into the integrated circuits of the processor 12, or loaded from memory 16, storage device 18, or network 24 or combinations thereof.

Bus 14 may represent one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computer system may include a variety of computer system readable media. Such media may be any available media that is accessible by computer system, and it may include both volatile and non-volatile media, removable and non-removable media.

System memory 16 can include computer system readable media in the form of volatile memory, such as random access memory (RAM) and/or cache memory or others. Computer system may further include other removable/non-removable, volatile/non-volatile computer system storage media. By way of example only, storage system 18 can be provided for reading from and writing to a non-removable, non-volatile magnetic media (e.g., a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 14 by one or more data media interfaces.

Computer system may also communicate with one or more external devices 26 such as a keyboard, a pointing device, a display 28, etc.; one or more devices that enable a user to interact with computer system; and/or any devices (e.g., network card, modem, etc.) that enable computer system to communicate with one or more other computing devices. Such communication can occur via Input/Output (I/O) interfaces 20.

Still yet, computer system can communicate with one or more networks 24 such as a local area network (LAN), a general wide area network (WAN), and/or a public network (e.g., the Internet) via network adapter 22. As depicted, network adapter 22 communicates with the other components of computer system via bus 14. It should be understood that although not shown, other hardware and/or software components could be used in conjunction with computer system. Examples include, but are not limited to: microcode, device drivers, redundant processing units, external disk drive arrays, RAID systems, tape drives, and data archival storage systems, etc.

It is understood in advance that although this disclosure may include a description on cloud computing, implementation of the teachings recited herein are not limited to a cloud computing environment. Rather, embodiments of the present invention are capable of being implemented in conjunction with any other type of computing environment now known or later developed. Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as Follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as Follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as Follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

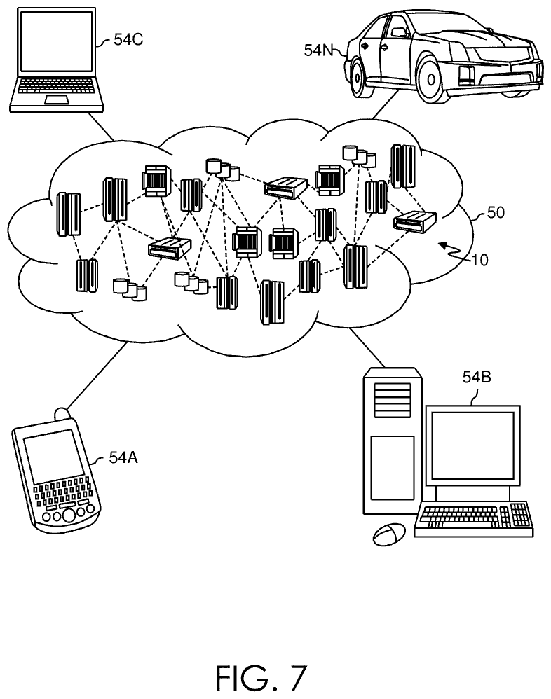

Referring now to FIG. 7, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 7 are intended to be illustrative only and that computing nodes 10 and cloud computing environment 50 can communicate with any type of computerized device over any type of network and/or network addressable connection (e.g., using a web browser).

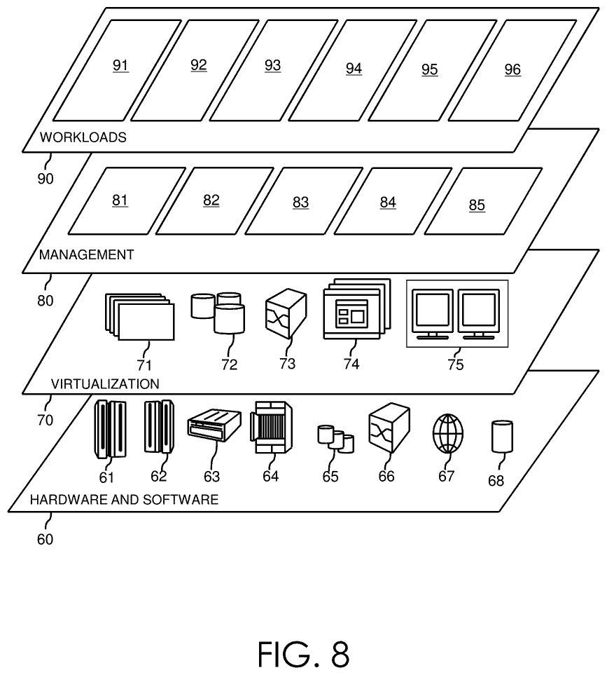

Referring now to FIG. 8, a set of functional abstraction layers provided by cloud computing environment 50 (FIG. 7) is shown. It should be understood in advance that the components, layers, and functions shown in FIG. 8 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and machine learning training processing 96.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, run concurrently, substantially concurrently, in a partially or wholly temporally overlapping manner, or the blocks may sometimes be run in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular embodiments only and is not intended to be limiting of the invention. As used herein, the singular forms “a”, “an” and “the” are intended to include the plural forms as well, unless the context clearly indicates otherwise. As used herein, the term “or” is an inclusive operator and can mean “and/or”, unless the context explicitly or clearly indicates otherwise. It will be further understood that the terms “comprise”, “comprises”, “comprising”, “include”, “includes”, “including”, and/or “having,” when used herein, can specify the presence of stated features, integers, steps, operations, elements, and/or components, but do not preclude the presence or addition of one or more other features, integers, steps, operations, elements, components, and/or groups thereof. As used herein, the phrase “in an embodiment” does not necessarily refer to the same embodiment, although it may. As used herein, the phrase “in one embodiment” does not necessarily refer to the same embodiment, although it may. As used herein, the phrase “in another embodiment” does not necessarily refer to a different embodiment, although it may. Further, embodiments and/or components of embodiments can be freely combined with each other unless they are mutually exclusive.

The corresponding structures, materials, acts, and equivalents of all means or step plus function elements, if any, in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A computer-implemented method of training a neural network for disease detection in a sample, comprising:
receiving gene expression data associated with a subject;
determining pair-wise similarities between genes in the gene expression data;
transforming the gene expression data into topological summaries based on the pair-wise similarities; and
training a neural network using a training set created based on the topological summaries.

2. The computer-implemented method of claim 1, further including:
receiving a new sample; and
inputting the new sample to the neural network, the neural network predicting the new sample's phenotype.

3. The computer-implemented method of claim 1, wherein the neural network includes a convolutional neural network.

4. The computer-implemented method of claim 1, wherein the pair-wise similarities include distance measures between pairs of genes in the gene expression data.

5. The computer-implemented method of claim 1, wherein the pair-wise similarities are used to create a point cloud, the point cloud used to transform the gene expression data into the topological summaries.

6. The computer-implemented method of claim 1, further including resampling data points based on the gene expression data, wherein the resampled data points are transformed into the topological summaries.

7. The computer-implemented method of claim 1, further including subsampling data points based on the gene expression data, wherein the subsampled data points are transformed into the topological summaries.

8. The computer-implemented method of claim 1, wherein the topological summaries are converted to a tensor and the tensor is fed into the neural network for training the neural network.

9. A system comprising:
a processor; and
a memory device coupled with the processor;
the processor configured to at least:
receive gene expression data associated with a subject;
determine pair-wise similarities between genes in the gene expression data;
transform the gene expression data into topological summaries based on the pair-wise similarities; and
train a neural network using a training set created based on the topological summaries.

10. The system of claim 9, wherein the processor is further configured to:
receive a new sample; and
input the new sample to the neural network, the neural network predicting the new sample's phenotype.

11. The system of claim 9, wherein the neural network includes a convolutional neural network.

12. The system of claim 9, wherein the pair-wise similarities include distance measures between pairs of genes in the gene expression data.

13. The system of claim 9, wherein the pair-wise similarities are used to create a point cloud, the point cloud used to transform the gene expression data into the topological summaries.

14. The system of claim 9, wherein the processor is further configured to resample data points based on the gene expression data, wherein the resampled data points are transformed into the topological summaries.

15. The system of claim 9, wherein the processor is further configured to subsample data points based on the gene expression data, wherein the subsampled data points are transformed into the topological summaries.

16. A computer program product comprising a computer readable storage medium having program instructions embodied therewith, the program instructions readable by a device to cause the device to:
receive gene expression data associated with a subject;
determine pair-wise similarities between genes in the gene expression data;
transform the gene expression data into topological summaries based on the pair-wise similarities; and
train a neural network using a training set created based on the topological summaries.

17. The computer program product of claim 16, wherein the device is further caused to:
receive a new sample; and
input the new sample to the neural network, the neural network predicting the new sample's phenotype.

18. The computer program product of claim 16, wherein the device is further caused to resample data points based on the gene expression data, wherein the resampled data points are transformed into the topological summaries.

19. The computer program product of claim 16, wherein the pair-wise similarities include distance measures between pairs of genes in the gene expression data.

20. The computer program product of claim 16, wherein the pair-wise similarities are used to create a point cloud, the point cloud used to transform the gene expression data into the topological summaries.
